(19) United States
(12) Patent Application Publication
(10) Pub. No.: US 2020/0352581 A1
(43) Pub. Date: Nov. 12, 2020

(54) OSTEOTOMY PROTECTION AND GUIDANCE DEVICE

(71) Applicants: MARTIN RUSSI, Montevideo (UY); JULIO EDUARDO AMAYA, Mendoza (AR)

(72) Inventors: MARTIN RUSSI, Montevideo (UY); JULIO EDUARDO AMAYA, Mendoza (AR)

(21) Appl. No.: 16/869,002

(22) Filed: May 7, 2020

(30) Foreign Application Priority Data
May 7, 2019 (UY) 38.218

(51) Int. Cl. A61B 17/17; A61B 90/00

(52) U.S. Cl. CPC A61B 17/17; A61B 2017/00424; A61B 2090/08021; A61B 90/08

(57) ABSTRACT

A protective device and guide for minimal invasive osteotomy during surgical procedures is disclosed, protecting tissues, muscles, tendons, and peripheral blood vessels adjacent to the bone structure, while allowing a spatial fixation of the device in order to free both hands of the healthcare professional in order to carry out the osteotomy without requiring additional staff.

### PRIOR ART

### Field of Invention

The present invention relates to the field of medical devices, means or arrangements, preferably in surgical procedures, more preferably osteotomies, and more particularly refers to a device that makes said procedures easier and improves the cutting or abrading of a bone piece which, unlike conventional devices, allows for greater cutting precision and greater protection of the tissues, nerves, muscles and peripherals present in the intervened bone part, providing correct and precise guidance of the cutting or abrading tool, thus allowing for the osteotomy in a minimally invasive manner.

### Prior Art Description

When a person usually requires surgical intervention in which it is necessary to cut some bone structure, or remove a tissue adjacent to said bone structure, a specialist surgeon must make a broad approach both to allow for good visualization of the operating field as well as for the protection of adjacent structures to avoid possible risks that would compromise patient health during surgery. In order to proceed, the surgeon must make considerable incisions in the patient's skin, muscles and other tissues, placing separators and protective material such as gauze and dressings, thus protecting noble structures such as nerves, which otherwise could be injured during the incisions. There is also the risk of cutting any blood vessel that could make the surgical intervention difficult.

There are currently different devices for carrying out osteotomy processes that allow the cutting of bone structures and/or the removal of tissue, which use protective elements to prevent damage to adjacent structures or organs. Such is the case of the device described in U.S. Pat. No. 8,062,300 B2, which is made up of an elongate main body comprising a proximal handle, a distal handle, a guide wire and a guide conduit; these last two elements inter-connect both proximal and distal handles. The proximal handle is interconnected to a guide wire, which is made up of a rigid proximal section and a flexible distal section, at the end of which there is a coupling element to allow the interconnection between the guide conduit and the guide wire.

Tissue modifying elements are positioned on one of the surfaces of the flexible distal section and act as the abrading element of the device. In order to prevent tissue-modifying elements from damaging structures or tissues before they are positioned in their working position, they are protected by a protective cover, preferably made of polymer material. The guide wire is attached at the other end to the distal handle, which has a tensioning element used to modify the tension level of the guide wire.

However, the device described above has the disadvantage of not being ergonomically designed, which makes it difficult for the medical team to use it. Another disadvantage is that the cut made by this device is by abrading and not by net cutting. This has the disadvantage that, being a very flexible device, only very small structures can be cut.

Another device is the one disclosed in U.S. Pat. No. 8,048,080 B2, which comprises a main body consisting of an elongated body, a handle, an actuator, tissue modification members and a protective surface. The elongated body has a proximal section and a distal section, which presents a window for the tissue modification members to come into contact with the tissue to be modified.

Before introducing the elongated body into the area of tissues to be modified, a guide member must be inserted, which will serve as a guide and support element for the main body; as the elongated body is introduced into the patient's body, the guide member is inserted into the central part of the elongated body; once the elongated body is placed in its working position, the end of the guide member which is free and outside the patient's body is held firmly in place; the handle is held firmly in place and, through the operation of the actuator, the tissue modification members begin to perform their abrading or cutting function. Once the procedure is completed, the main body and the guide member are removed from the patient's body.

The device described above, due to its configuration and design, requires at least two people to operate properly, since it requires the opposite end of the guide member to be held firmly, either by an anchoring mechanism or by a user's hand. Like the device in U.S. Pat. No. 8,062,300B2, this device cuts by abrading and not by net cutting and furthermore does not have a mechanism that allows grading and measuring the cut in any spatial plane.

Another device is the one disclosed in U.S. Pat. No. 6,423,080 B1, which describes a device used for the fastening or securing of structures and/or organs, which comprises a pair of positioning members, which are made up of a handle, a guide wire, a support, a cutting element and an actuator. The handle has a hollow tubular body in whose upper section an actuator is located and, in its lower section, a support that keeps the guide line in position. The handle has a rough surface on its outside face, which increases the grip of the handle.

The guide wire has a straight proximal section, a curved middle section and a straight distal section. In an additional mode of the device, the straight distal section is oriented at a 90° angle to the position of the straight proximal section. In addition, the guide conduit is hollowed out to place the cutting element, which is also hollowed out to allow the guide member to pass through. The cutting element is used to open a small opening in the tissue to allow the positioning element to pass through.

However, it has the disadvantage that it is only designed to position a cord or sling over a tissue or structure that requires it, and not to cut bone structures. In turn, the removal of the two positioning members and the guide member is required for the placement of the cord or sling, which makes it considerably more difficult to operate.

On the other hand, U.S. Pat. No. 9,545,260 B2 describes a device for cutting at least a pair of assembly elements consisting of a first main body and a second main body joined together at their proximal and distal ends; a fastening element inserted at the distal end of both first and second main bodies respectively, which is used to hold them together; a threading element which is inserted in its inner section from the inter medial section of that device, continues to its proximal end and returns to its intermediate section from the opposite end; and, a multi-function element which is located inside the threading element.

However, this conformation does not allow the performance of minimally invasive osteotomies with drill or reamer, which allow a faster cut, a smaller incision without the need to take off all the tissues surrounding the bone even allowing incomplete osteotomies such as the Reverdin-Isham technique.

While the aforesaid documents have proven to work in practice, there are still a number of drawbacks. As mentioned above, they involve invasive interventions that can damage and compromise tissues, muscles or blood vessels peripheral to the bone structure. Moreover, in addition to being invasive, no control of cutting or abrading is allowed on the bone, generating a possible problem if the cut is greater than it should be. In addition, the vast majority requires at least two people to carry out the osteotomy process, so both people must be coordinated to successfully perform the surgery and avoid any damage to the patient.

As a result of the above, Published Patent Document No. U.S. 2014/0343555, from the same owner of this application, has been developed to refer to techniques for designing and constructing devices used in surgical interventions, through less invasive techniques with protection and cutting guidance that generally comprise a pair of assembly components formed by a first main body and a second main body joined together in their middle, or intersection area, and at the distal ends of the handles forming a cross-fastening type system. This requires the holding of the tool by one of the doctor's hands, but could be fixed by a spatial support structure that is independent of the tool and must be connected, in some way, to one of the first and second bodies that articulate with each other. This spatial fixation, however, has not demonstrated the expected results.

Although the tool of the same applicant has demonstrated improvements over the prior art, the present inventor and owner of Application U.S. 2014/0343555, has continued to research and develop in the field of the art in order to further minimize the invasion during the intervention and to obtain a much better spatial fixation of the tool, in order to free both hands of the practitioner and make the tasks easier to perform the osteotomy in a much more precise, practical, fast and efficient manner.

### BRIEF DESCRIPTION OF THE INVENTION

It is therefore an object of the present invention to provide a new protection and guidance device for minimally invasive osteotomy that facilitates the tasks of cutting and/or abrading bone structures.

It is another object of the present invention to provide a protective and guiding device that allows protecting tendons, muscles, tissues and/or peripheral blood vessels adjacent to the bone structure to be intervened, thus minimizing the damage in the intervened area.

It is yet another object of the present invention to provide a protective and guiding device that allows greater stability of the device during osteotomy, in order to improve incision precision and preventing the device from moving and generating unintentional accidents.

It is furthermore an object of the present invention to provide a protective and guiding device that is fixed in such a way as to allow the release of the practitioner's hands, so the practitioner can perform the osteotomy process with only one hand.

It is another object of the present invention to provide a protective and guiding device that allows the osteotomy to be performed by one professional, and not at least two as required in prior art.

It is still another object of the present invention to provide a protective and guiding device, provided with at least one element for spatial positioning which allows for incision graduation of the bone structure in the three planes: sagittal, frontal and rotational.

It is still another object of the present invention to provide an osteotomy protection and guidance device, comprising at least one handle; at least one intermediate element connected to said handle and provided with an opening for a surgical intervention device; at least one insertion and protection tip connected to said intermediate element; at least one positioning element to pass through; and at least one fixation nail passing through said positioning element in a guided manner.

### BRIEF DESCRIPTION OF THE FIGURES

For greater clarity and understanding of the present invention, it has been illustrated using several figures, whereby the invention has been represented in one of the preferred embodiments, all by way of example, wherein:

FIG. 1 is a perspective view of a preferred first form of implementation of the osteotomy protection and guidance device, in accordance with the present invention;

FIG. 2 is a more detailed perspective view of the device in FIG. 1;

FIG. 3 is a perspective view of the device in the present invention, wherein at least one positioner is provided and where a possible application is shown;

FIG. 4 shows another perspective view of the device in FIG. 3;

FIG. 5 shows a perspective and partial view of a second preferred form of realization of the device in the invention, wherein the provision of a positioner of the space type may be observed;

FIGS. 6 and 7 show views of possible applications of the device in FIG. 5, in the metatarsals of the foot, according with the present invention;

FIGS. 8 to 10 show views of different elements forming a positioning element for the device of the present invention of FIG. 5; and

FIG. 11 shows a perspective view of one of the elements of the positioner mounted on a handle of the device, in accordance with the present invention.

### DETAILED DESCRIPTION OF THE INVENTION

Referring now to the figures, it is noticed that the invention consists of a new protection and guidance device for osteotomy that minimizes invasion during surgical intervention, protecting tissues, muscles, tendons, peripheral blood vessels adjacent to the bone structure intervened, while allowing a spatial fixation of the device in order to free both hands of the healthcare professional in order to carry out the osteotomy without requiring additional auxiliary personnel. In addition, it provides safe protection to nervous structures.

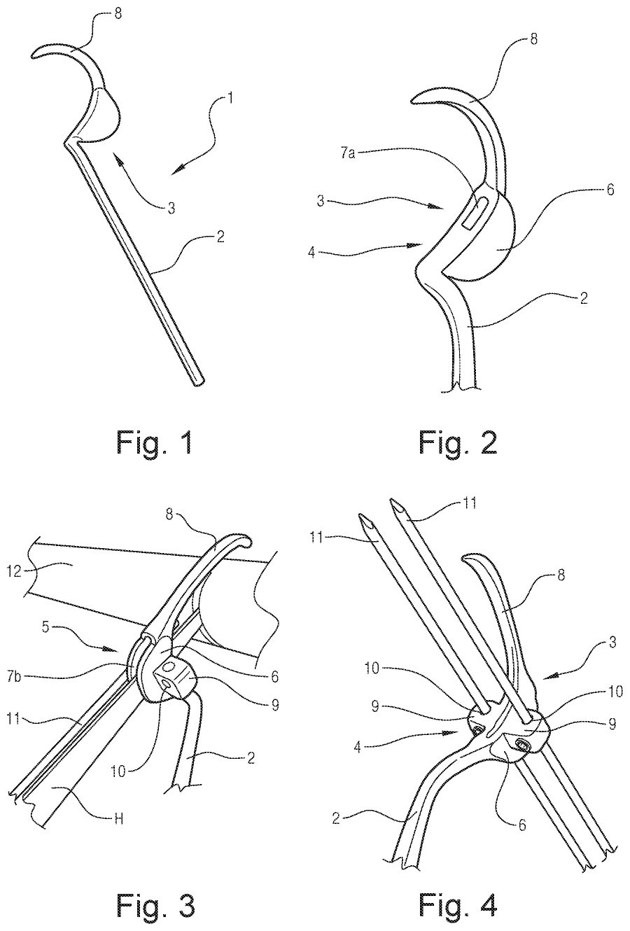

Therefore, and in accordance with FIGS. 1 to 4, the osteotomy protection and guidance device of the present invention is indicated by the general reference 1 and comprises in a first preferred form of execution at least a handle 2, which extends in the form of a bayonet connecting with an intermediate element 3. It should be noted that the handle 2 may present different lengths, which will adapt to the different needs and requirements depending on each type of surgical intervention, as well as, the handle may present a section of variable length, which may take place by presenting at least two parts which can be dismantled from each other, allowing the variation in the total length of the handle.

The intermediate element 3 is connected to the handle 2 and comprises an internal face 4, an external face 5, lateral “ears” or sides 6 and a through-hole or central opening 7a that operatively connects both faces 4, 5 allowing the guided passage of a surgical intervention device that may be any related cutting or abrading tool, in order to be used for the osteotomy. The guidance of the tool is also improved by the containment that the ears 6 will provide to the cutting or abrading tool that will pass through the channel 7b defined between them and that can be on the side of the internal face, as well as on the external face. A longitudinally defined channel 7b along the development of the intermediate element 3 allows for easier movement or guided pivoting of the tool up and down to avoid false movements and thus improve the osteotomy process to be success.

It should be noted that, as mentioned above, the through-hole 7b of the intermediate element faces the area to be worked on and allows the introduction of at least one drill of a given calibre, so the cut is directed in the plane that determines the internal face of the curved end in the form of a cutting guide.

Moreover, the present invention is provided with at least one insertion and protection tip 8 connected to said intermediate element 3. Said insertion and protection tip 8 presents a curved development, which is inserted in the body of the patient, surrounding the bone structure to be intervened, thus protecting the tissues, muscles, blood vessels and peripheral related to the same from possible involuntary accidents. The insertion tip 8 will protect any tendon, ligament, muscle, blood vessel, etc., that is outside its radius, while internally it will isolate the bone structure to be intervened from the rest. The radius of curvature of the insertion tip 8 can vary depending on the bone structure to be operated on and allows it to be surrounded as the osteotomy is performed. This has a positive advantage over the prior art, as it allows the areas peripheral to the bone structure to be protected at the same time as the tool is advanced during the osteotomy.

By presenting at least, but not limited to the invention, one insertion tip 8, the movement of the device during the development of the osteotomy is greatly facilitated, since there are no two pieces that should move carefully and simultaneously to continue with the osteotomy process, as occurs in the Publication of Patent U.S. 2014/0343555 of the same owner.

On the other hand, in order to improve the osteotomy process and provide safety to the practitioner and consequently to the patient, at least one positioning element is provided allowing for spatial fixation of the device to free both hands from the practitioner. Wherein, the said positioner element 9 comprises at least a cube-shaped positioner structure provided with through-holes 10 for the guided pass through of at least one fixing element 11, which may be a kirschner nail or “schanz” fixation structures well known in the art and which will not be described in detail for such reasons.

The positioner 9 is connected to at least one of the ears or sides 6 of the intermediate element 3 by welding, not being a limit for the invention since other types of related linkage and union can be considered and/or used without any inconvenience. Although, in FIGS. 3 and 4, the positioner element 9 arranged in both sides 6 has been illustrated, this does not imply that the invention is limited to it, but it can also be the case of presenting a single positioner cube 9 in one side 6 of the intermediate element 3.

In this way, the positioning element or elements 9 in conjunction with the nails 11, allow the fixation of the device in the invention completely freeing both practitioner's hands, who will be able to perform the osteotomy without requiring additional personnel. At the same time, they not only free the hands but also allow the control of the device under radiological vision, before acting the drill in its interior and being able to make any modification if the surgeon considers it so (predetermined zone of the cutting action). As may be seen in FIG. 3, there is, as an example, a bone structure 12 which is protected and surrounded by the insertion and protection tip 8, and which is in turn fixed by a fixation nail 10 driven into it. Alternatively, the device can be rotated to facilitate this task, with the tip 8 rotating to protect the peripheral tissues, muscles, etc., which are in or near the bone structure being operated on. In order to do this, the device must not be nailed to the patient and must be controlled with the hand of the professional or assistant.

As aforesaid, the positioner element 9 allows spatially fixing the device of the invention freeing both hands of the professional. The practitioner thus has complete freedom to carry out the surgical intervention without requiring additional auxiliary personnel to assist him/her with holding the device. This translates into greater ease in fixing the device and continuing with the intervention in a much more practical, fast and efficient way, being this a great advantage in comparison to devices in prior art that do not allow carrying out such actions.

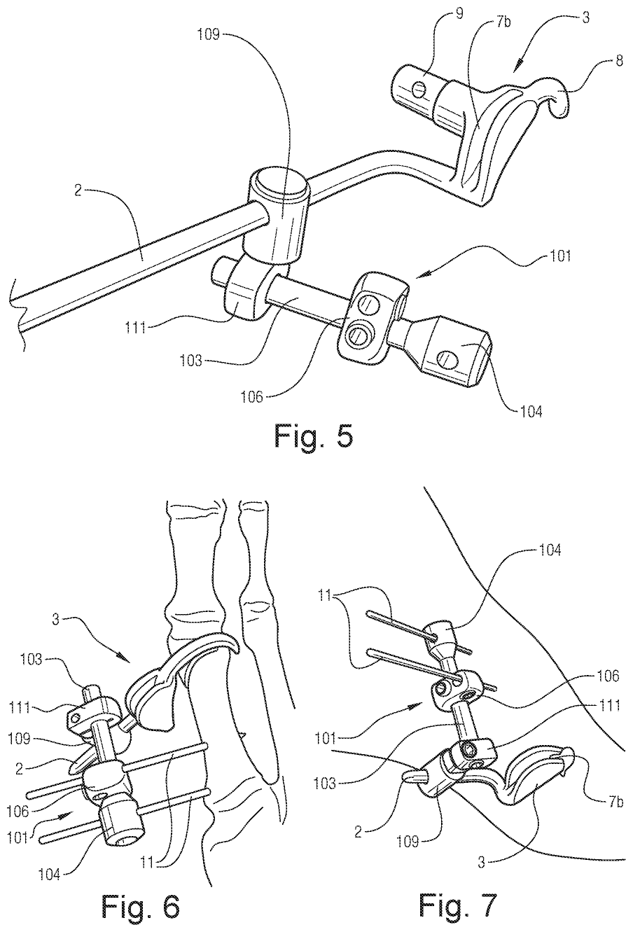

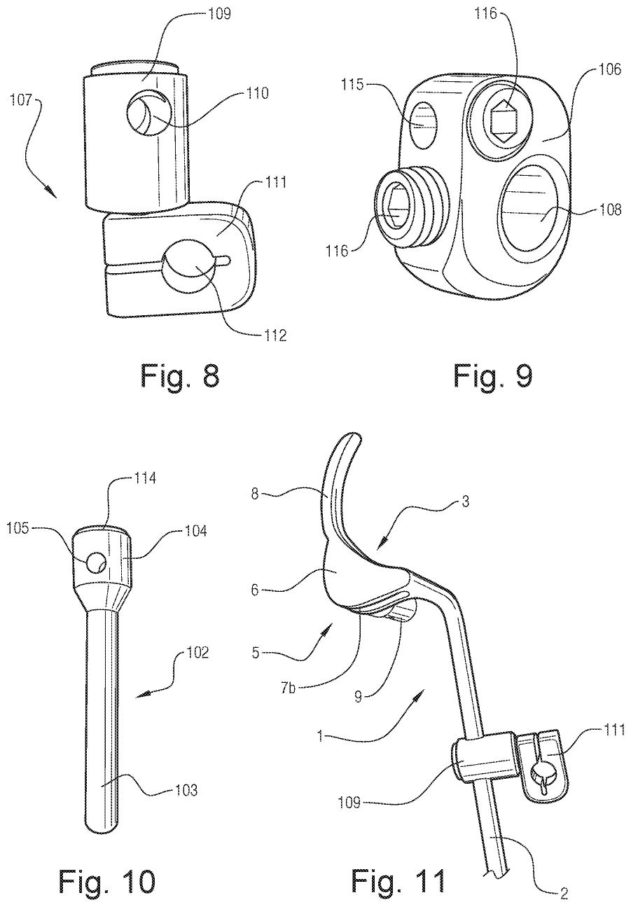

In a second preferred embodiment, FIGS. 5 to 11, there is a positioner element 101 which comprises at least one positioner pin 102 with a shaft 103, a locking screw 114 and a head 104 with at least one through-hole 105; at least one guide positioner 106; and at least one fixing bracket 107. One pair of holes 108 for the passage of the shaft 103 of the positioner pin and at least one hole 115 for the passage of the shaft at least one fastening pin 11, as better illustrated in FIGS. 6 and 7, being at the same time, a pair of screws 116 for blocking the structures passing through it.

On the other hand, the fixing bracket 107 comprises a fastening element 109, which can be a double cylinder with a hole 110 and a supporting element 111 also with at least a through-hole 112 and a locking screw (not shown) which allows both the handle 2 and the pin 102 to be locked permanently. It should be noted that the fixing bracket 107 is selectively mounted and fixed to said handle 2 through said fastening element 109, while said shaft 103 of the positioning pin 102 passes through the through-hole 112 of the supporting element 111 and is fixed to said handle and pin by the arrangement of said locking screw 113. In other words, the locking screw (not shown) is attached to both the fastening element 109 and support 111 by a threaded arrangement that secures the two cylinders and closes the gap formed in the support 111 by closing the through-hole 112.

In an alternative case of the invention, but not limited to it, both positioning elements 9 and 101 can be used as better illustrated in FIGS. 5 and 11. In this way, once the incision is made and the periosteum is removed, the healthcare professional places the device by taking it by the handle and making a movement in such a way that the insertion and protection tip is positioned between the soft tissues and the bone to be cut. Using the positioner element and the fastening elements to the bone, the ehalthcare professional stabilizes the device and establishes the cutting plane. The drill or reamer is then introduced through hole 7b (previously guided by channel 7a) in the intermediate element and the incision is made in the plane determined by the ears or sides of the intermediate element.

The arrangement of the positioner element 109 allows extending the device fixation radius in the invention even more, providing at the same time, more space and comfort when operating and undergoing a surgical procedure.

On the other hand, the device of the invention can be made of stainless steel, this not limiting the invention given any other compatible material for surgical use may be used that can diminish production costs and even its manufacture in disposable materials, such as for example plastics or other polymers, but knowing that the tip 8 must be manufactured of a material resistant to the top of the reamer or drill. Combined materials can also be used for different parts of the device if necessary, where they can be considered and used without any inconvenience.

In this way, the osteotomy protection and guidance device of the present invention is formed and manufactured, which can be spatially fixed to free both hands of the healthcare professional, allowing him to carry out the osteotomy without requiring additional personnel and to perform the intervention in a much more practical, fast and efficient way. At the same time, the invasion during the intervention is minimized and the tendons, muscles, tissues and/or peripheral blood vessels adjacent to the bone structure are protected.

When this device is used to cut bone structures, it includes the positioning element for spatial positioning allowing for the healthcare professional to orient, measure and graduate the incision of the bone structure in three spatial planes: frontal, sagittal and rotational. It should be noted that the curvature of the insertion and protection tip follows the bone contour of the metatarsals for use in minimally invasive foot surgery. In an additional embodiment of the present device, the tip curvature 8 follows the bone contour of the tibia for use in tibial proximal metaphyseal, diaphyseal or distal metaphyseal osteotomy. Additionally, the curvature follows the bone contour of the femur for use in proximal metaphyseal, diaphyseal, or distal metaphyseal osteotomy.

It is emphasized that the structure presenting the device for surgical procedures object of the present invention allows its use in long bone osteotomies, mainly femur and tibia; for procedures such as bone elongations, axial corrections of members; as well as osteotomy of flat bones such as the iliac, ischium and pubis.

As an expert may note, the system found in U.S. 2014/0343555, patent owner also owning the subject application, provides a device consisting of two parts or pieces forming a kind of “clamp” whose distal ends are inserted into the patient, while the proximal ends are connected to each other in order to close, as mentioned in the prior art section herein. However, this design and configuration shows much greater invasiveness than the one proposed by the present invention. Furthermore, it has been demonstrated in practice that the device in U.S. 2014/0343555 does not provide the required stability and fixation and therefore, the practitioner must use at least one hand to hold the device steady during the osteotomy procedure.

Taking into account the defects of the previous technique, the present invention allows solving the inconveniences of the prior art by providing a device for minimally invasive surgical procedures, which can be used in osteotomy and/or tissue removal procedures, in a practical and simple manner. It reduces to a minimum the time of surgical intervention and the internal damage generated to the patient and presents an ergonomic design and construction to facilitate its manipulation by the medical team. Likewise, it provides an adequate and correct spatial fixation, so the professional has both hands free to carry out the osteotomy without requiring additional auxiliary personnel. At the same time, the related tool used is properly guided to avoid unintentional damage in peripheral areas. The entire invention provides greater safety both for the healthcare professional, in order to carry out the osteotomy correctly and efficiently without damage, and for the patient due to the minimally invasive procedure.

Although the above description has described and shown preferred embodiments of the present invention, it should be emphasized that numerous modifications to the present invention are possible, without departing from the true scope of the invention, such as modifying the configuration of the fastening handles and coupling elements, modifying the profile of the various elements that make up the present device, modifying the fastening element, etc.

1. An osteotomy protection and guidance device comprising:
at least one handle;
at least one intermediate element connected to that handle and provided with an opening for the passage of a surgical device;
at least one insertion and protection tip connected to that intermediate element;
at least one positioning element; and
at least one fastening pin that passes through the positioner in a guided manner.

2. The osteotomy protection and guidance device in accordance with claim 1, wherein the at least one positioning element comprises at least one positioning cube having through-holes for the passage of at least one nail and which is connected to at least one side of said intermediate element.

3. The osteotomy protection and guidance device in accordance with claim 2, wherein the positioner cube is connected to the side of the intermediate element.

4. The osteotomy protection and guidance device in accordance with claim 1, wherein the positioner element comprises:
at least one positioning pin having a shaft and head with at least one guide through-hole;
at least one guide positioner; and
at least one fixing bracket.

5. The osteotomy protection and guidance device in accordance with claim 4, wherein the guide positioner is selectively mounted on a section of said positioner pin and comprises at least one prismatic body with a plurality of through-holes on each of its faces.

6. The osteotomy protection and guidance device in accordance with claim 4, wherein the positioner pin axis passes through a matching pair of said guide positioner holes, while at least one other hole passes through at least one fixing pin.

7. The protective and guiding device for osteotomy in accordance with claim 4, wherein the fixing support comprises a fixing element provided with a hole and a support element, also provided with at least one through-hole and connected to the fixing element.

8. The osteotomy protection and guidance device in accordance with claim 1, wherein said fixing support is selectively mounted and fixed in said handle through said fixing support, while said positioner pin axis passes through the through-hole of the fixing support element, thus being fixed through said fixing support.

9. The osteotomy protection and guidance device in accordance with claim 1, further comprising a curved development of the insertion and protection tip.
